(19) United States
(12) Patent Application Publication
(10) Pub. No.: US 2009/0157837 A1
(43) Pub. Date: Jun. 18, 2009

(54) NDMA SOCKET TRANSPORT PROTOCOL

(75) Inventors: Robert J. Hollebeek, Berwyn, PA (US); Frank Paul Hammitt, Knoxville, TN (US)

Correspondence Address: RATNERPRESTIA, P.O. BOX 980, VALLEY FORGE, PA, 19482, US

(73) Assignee: The Trustees of the University of Pennsylvania, Philadelphia, PA (US)

(21) Appl. No.: 12/372,976

(22) Filed: Feb. 18, 2009

Related U.S. Application Data
(63) Continuation of application No. 10/559,060, filed on May 2, 2006, now abandoned.

(30) Foreign Application Priority Data
Jun. 4, 2004 (US) PCT/US04/17847

(51) Int. Cl. G06F 15/16; G06F 17/30

(52) U.S. Cl. 709/206; 707/104.1; 707/E17.031

(57) ABSTRACT

Data transferred between devices 12/16 is formatted in accordance with a multi-layered data structure. One layer 66 of the multi-layered data structure includes a socket protocol. A layer 68 within the layer 66 includes a header. A payload layer 72 is included within the layer 68. The header includes information indicative of the type of data in the payload layer 72 and enables multiple data types to be transferred in their original format without conversion. This multi-layered data structure may provide DICOM interactions with medical devices within a hospital secure network 14 to be coupled with external communications mechanisms 16 that can acquire or store NDMA content while maintaining the integrity of the hospital/clinic network security and incorporating strong firewall-like protections. The layer 66 of the multi-layered data structure may further include routing information used to route data received at a destination port 5007 to another port 5005.

### CROSS REFERENCE TO RELATED APPLICATIONS

The present application claims priority to U.S. Provisional Application No. 60/475,940, filed Jun. 4, 2003, entitled “NDMA SOCKET TRANSPORT PROTOCOL,” which is hereby incorporated by reference in its entirety. The subject matter disclosed herein is related to the subject matter disclosed in U.S. patent application serial number (Attorney Docket UPN-4380/P3179, filed on even date herewith and entitled “CROSS-ENTERPRISE WALLPLUG FOR CONNECTING INTERNAL HOSPITAL/CLINIC IMAGING SYSTEMS TO EXTERNAL STORAGE AND RETRIEVAL SYSTEMS”, the disclosure of which is hereby incorporated by reference in its entirety. The subject matter disclosed herein is also related to the subject matter disclosed in U.S. patent application serial number (Attorney Docket UPN-4382/P3189, filed on even date herewith and entitled “NDMA SCALABLE ARCHIVE HARDWARE/SOFTWARE ARCHITECTURE FOR LOAD BALANCING, INDEPENDENT PROCESSING, AND QUERYING OF RECORDS”, the disclosure of which is hereby incorporated by reference in its entirety. The subject matter disclosed herein is further related to the subject matter disclosed in U.S. patent application serial number (Attorney Docket UPN-4383/P3190, filed on even date herewith and entitled “NDMA DATABASE SCHEMA, DICOM TO RELATIONAL SCHEMA TRANSLATION, AND XML TO SQL QUERY TRANSLATION”, the disclosure of which is hereby incorporated by reference in its entirety.

### FIELD OF THE INVENTION

The present invention generally relates to a multi-layered data structure for transferring data between medical facilities and external services and, more particularly, to a four layer nested structure for transferring data between DICOM or HL7 compatible imaging systems and NDMA compatible storage systems.

### BACKGROUND

Prior systems for storing digital mammography data included making film copies of the digital data, storing the copies, and destroying the original data. Distribution of information basically amounted to providing copies of the copied x-rays. This approach was often chosen due to the difficulty of storing and transmitting the digital data itself. The introduction of digital medical image sources and the use of computers in processing these images after their acquisition has led to attempts to create a standard method for the transmission of medical images and their associated information. The established standard is known as the Digital Imaging and Communications in Medicine (DICOM) standard. Compliance with the DICOM standard is crucial for medical devices requiring multi-vendor support for connections with other hospital or clinic resident devices.

The DICOM standard describes protocols for permitting the transfer of medical images in a multi-vendor environment, and for facilitating the development and expansion of picture archiving and communication systems and interfacing with medical information systems. It is anticipated that many (if not all) major diagnostic medical imaging vendors will incorporate the DICOM standard into their product design. It is also anticipated that DICOM will be used by virtually every medical profession that utilizes images within the healthcare industry. Examples include cardiology, dentistry, endoscopy, mammography, ophthalmology, orthopedics, pathology, pediatrics, radiation therapy, radiology, surgery, and veterinary medical imaging applications. Thus, the utilization of the DICOM standard will facilitate communication and archiving of records from these areas in addition to mammography. Therefore, a general method for interfacing between instruments inside the hospital and external services acquired through networks and of providing services as well as information transfer is desired. It is also desired that such a method enable secure cross-enterprise access to records with proper tracking of accessed records in order to support a mobile population acquiring medical care at various times from different providers.

In order for imaging data to be available to a large number of users, an archive is appropriate. An archive that has been developed is the National Digital Mammography Archive (NDMA) which stores digital mammography data. The NDMA acts as a dynamic resource for images, reports, and all other relevant information tied to the health and medical record of the patient. Also, the NDMA is a repository for current and previous year studies and provides services and applications for both clinical and research use. The development of such a national breast imaging archive may very well revolutionize the breast cancer screening programs in North America. The privacy of the patients is a concern. Thus, the NDMA ensures the privacy and confidentiality of the patients, and be compliant with all relevant federal regulations.

To facilitate distribution of, and access to this imaging data, DICOM compatible systems should be coupled to the NDMA. To reach a large number of users, the Internet would seem appropriate; however, the Internet is not designed to handle the protocols utilized in DICOM. Therefore, while NDMA supports DICOM formats for records and supports certain DICOM interactions within the hospital, NDMA uses its own protocols and procedures for file transfer and manipulation.

Thus, a need exists for a mechanism that couples DICOM compatible systems to the NDMA in a way that provides privacy and security, that does not hamper operations on the DICOM side, that provides encryption on the external network (NDMA) side, that provides strong authentication and external management, and that can efficiently transfer large amounts of data between the DICOM system and the NDMA.

### SUMMARY OF THE INVENTION

Data transferred between DICOM compatible devices located at a hospital or clinic and external NDMA compatible storage and retrieval systems are formatted in accordance with a four layer socket protocol (data structure). The first layer of this multi-layered data structure includes an NDMA socket protocol. The second layer is nested within the first layer and includes an NDMA header. The third layer is nested within the second layer and includes XML text. The fourth layer is nested within the third layer and includes DICOM, or other binary, related data This multi-layered data structure provides DICOM interactions with medical devices within the hospital secure network to be coupled with external communications mechanisms which can acquire or store NDMA content while maintaining the integrity of the hospital/clinic network security and incorporating strong firewall-like protections.

The multi-layered socket protocol supports encryption of all external traffic to protect patient privacy, including encryption of medical records transferred via external networks. To maintain security within the hospital private network, all administrative functions and connections to the communication devices are secured. This is accomplished with a secure, protected web interface. The web interfaces support the use of strong authentication via smart cards and security certificates.

In an exemplary embodiment of the present invention, a multilayered data structure for communicating binary image data between a device that generates the binary image data and a remote NDMA archive system for storage of the binary data, includes four layers. The first layer includes a socket protocol. The second layer is nested within the first layer and includes a national digital mammography archive (NDMA) header. The third layer is nested within the second layer and includes extensible markup language (XML) text. The fourth layer is nested within the third layer and includes the binary image data.

The present invention also includes a method for transferring binary image data between (in either direction) a digital imaging and communications in medicine (DICOM) compatible device and a storage device. In an exemplary embodiment, the binary image data comprises either DICOM related data or a binary payload. Such a method in accordance with the invention comprises the steps of:

opening a socket and sending a socket protocol header indicating a total number of bytes to follow;

sending a first NDMA header for content type XML, each NDMA header containing version and length specifiers;

sending an XML message containing message identifiers, requested actions, and sender and receiver specifications;

sending a second NDMA header for content type binary image data; and

sending a binary payload containing the binary image data.

Other features and advantages of the present invention will be apparent from the following detailed description.

### BRIEF DESCRIPTION OF THE DRAWINGS

FIG. 1 is a block diagram of firewalled hospital systems coupled to a WallPlug via a DICOM compatible network, and an archive coupled to the WallPlug via a virtual private network in accordance with an exemplary embodiment of a system implementing the present invention;

FIG. 2 is a block diagram showing the WallPlug comprising a first portal coupled to the DICOM compatible network, a second portal coupled to the virtual private network, and the two portals coupled together via a private secure network in accordance with an exemplary embodiment of a system implementing the present invention;

FIG. 3 is a more detailed block diagram of the WallPlug showing software and hardware components in accordance with an exemplary embodiment of a system implementing the present invention;

FIG. 4 is a diagram of the four nested layers of the National Digital Mammography Archive (NDMA) socket transport protocol in accordance with an exemplary embodiment of the present invention;

FIG. 5 is a block diagram of software components in the National Digital Mammography Archive (NDMA) utilized to transfer data to and from the WallPlug in accordance with an exemplary embodiment of a system implementing the present invention; and

FIG. 6 is a block diagram of the NDMA system in accordance with an exemplary embodiment of a system implementing the present invention.

### DESCRIPTION OF EMBODIMENTS OF THE INVENTION

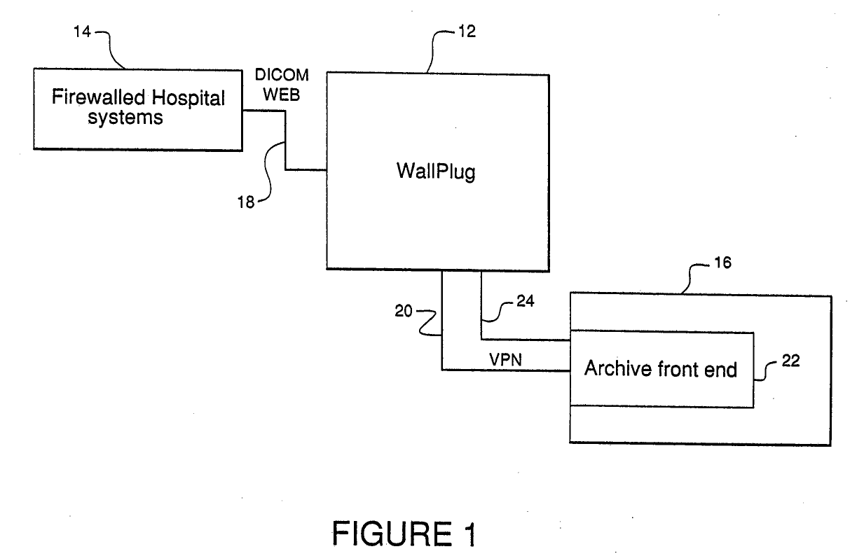

FIG. 1 illustrates a system for implementing the multi-layered socket protocol in accordance with the present invention. In accordance with the invention, a multi-layered socket protocol is used to transfer information between the WallPlug 12 and the NDMA 16. The NDMA 16 uses a socket protocol between a sender process (MAQ) and a receiver process (MAQRec) to transfer queues of medical image and records files. Both processes are multi-threaded and provide extensive error handling and recovery. The sender process handles scheduling and batching of files in its input queue. Each sender has a specific input queue, socket port number and destination IP address. Receivers have socket port numbers, and output queue directories. In one embodiment, the multi-layered socket protocol is compatible for use with both WINDOWS® and LINUX®/UNIX® operating systems providing machine operating system independent information transfer.

FIG. 1 is a simplified block diagram of the WallPlug 12 coupled to firewalled hospital systems 14 and coupled to an archive front end 22 of an archival and retrieval system 16 in accordance with an exemplary embodiment of a system implementing the protocol of the present invention. The multi-layered socket protocol is used to transfer information between the WallPlug 12 and the NDMA 16 via a virtual private network (VPN) 20 or 24. The WallPlug 12 is coupled to the firewalled hospital systems 14 via a TCPIP compatible network 18. The network 18 can be any appropriate TCPIP compatible network such as a DICOM compatible network, an HL7 compatible network, an Internet or Web compatible network, or the like. The VPN compatible network 20 or 24 can be any appropriate VPN.

The network 18 provides virtual secured access, such as DICOM, HL7, and/or web access, from enabled hospital/clinic medical devices, smart cards, or certificate enabled systems through the combination of servers in the WallPlug 12. The WallPlug 12 provides secured access to test records, patient records, administrative control, or a combination thereof. The WallPlug 12 presents a secure web user interface and a DICOM hospital instrument interface on the hospital side and a secure connection to the archive on the VPN side. The WallPlug 12 makes no assumptions about external connectivity of the connected hospital systems 14 and can operate without any connectivity other than the VPN 20. In one embodiment, the WallPlug 12 comprises an external connection to a second VPN 24 for providing communications redundancy, hardware testing, and/or management in the event of a failure.

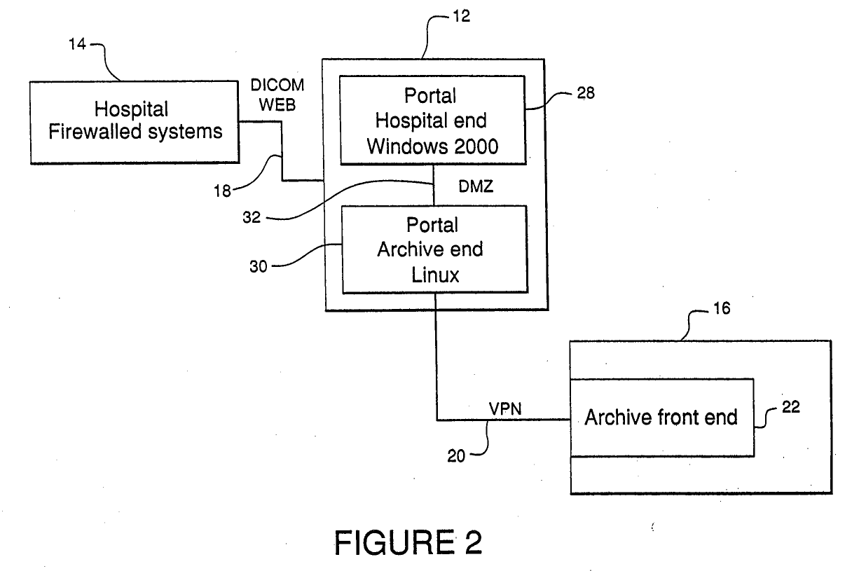

FIG. 2 is a block diagram of the WallPlug 12 comprising a first portal system (portal) 28 coupled to the DICOM compatible network 14 and a second portal 30 coupled to the virtual private network 20 in accordance with an exemplary embodiment of a system implementing the protocol of the present invention. The multi-layered socket protocol is implemented everywhere except between the WallPlug portal 28 and the hospital system 14 unless the hospital device is NDMA compatible. The multi-layered socket protocol is used for all communications between the portal 28 and the portal 30 of the WallPlug 12. The two portals 28, 30 are coupled together via a private secure network 32. The WallPlug 12 provides the on-site hospital/clinic medical interface to external services and systems. Generally, the WallPlug 12 can be constructed from any pair of servers or special hardware with two isolated processor units. In an exemplary configuration, each portal may comprise an IBM server; each portal having two CPUs, two redundant power supplies, and a management interface. The two management interfaces can be linked together to an ASM (system management device) which can be used to externally monitor the two systems. The portals 28, 30 do not necessarily need to operate under the same operating systems. For example, the exemplary depiction shown in FIG. 2 shows the portal 28 operating under WINDOWS® 2000 and the portal 30 operating under LINUX®. It is to be understood that the portals 28, 30 can operate under other combinations of operating systems (including the same operating system), and should not be limited to the exemplary operating systems depicted in FIG. 2. The portals 28, 30 are the sole hardware interface between the hospitals systems 14 and the distributed storage and retrieval services systems 16. The portals 28, 30 are easily deployed and maintained, and provide secure encrypted links between the hospital systems 14 and the archive systems 16.

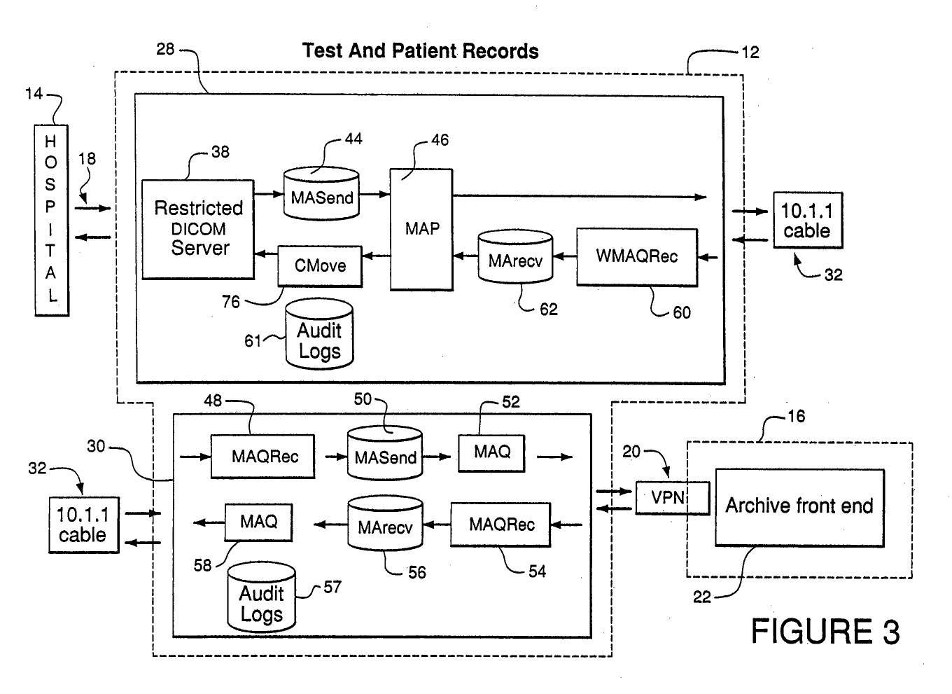

FIG. 3 is a block diagram of the WallPlug 12 showing software and hardware components utilized for test records and patient records in accordance with an exemplary embodiment of a system implementing the protocol of the present invention. The multi-layered socket protocol is used to transfer information internally between the various software components shown in FIG. 3. Data flows between the hospital 14 and the archive 16 and returns. Implementation utilizes generalized senders and receivers along with the MAP 46 which is the primary traffic manager, logger and scheduler. The MAQ 52 is a sender that takes files from a worklist, and sends them to a receiver. The MAQRec 54, on the other hand, is a receiver that receives files and places them in a queue. Both processes log all actions in, e.g., audit log 57, and use the NDMA protocols.

As shown in FIG. 3, the portal software on the hospital side is responsible for running the DICOM server 38, and for transferring files from the DICOM server 38 to the private network 32 linking the two portions of the WallPlug 12. The software which does this includes software called MAP that interfaces to the DICOM server 38 and includes DICOM test and diagnostic software, a queue manager that watches for new files in the input MASend directory 44, and mechanisms to transfer the files via sockets on the crossover cable 32 to the backend portal 30. All activities that move or manipulate files on each portal 28, 30 are logged in two databases, one for operational messages and one which audits the movement of all files. The latter is required for HIPAA (Health Insurance Portability and Accountability Act) compliance and both are forwarded to the archive 16 periodically and entered into a master database. The database 61 represents all databases for the portal 28 and the database 57 represents all databases for the portal 30. The queue software detects errors and will retry the transmission to the next stage as necessary. Sufficient local cache can be implemented on the system to allow autonomous operation for days should downstream elements be temporarily inoperable.

The portal software of portal 28 also assists in the transfer of records back to the hospital. An application using the socket protocol (WMAQRec 60) running on the private cable receives files from the backend portal 30 and stores them in the MArecv directory 62. The MAP 46 software receiver components transfer and log these files to approved locations using a CMOVE 76 through the DICOM server 38. Again, all movement of files is logged through the protocol and the logs are forwarded to the archive 16 periodically.

All senders and receivers provide extensive logs of all transactions, errors, and file movement. Log files locations can be externally specified. All log files have a control which can be used to enable/disable multiple levels of output from level 0 (summary and error logging only) to higher integer values providing more detailed information. Error output is standardized to contain debug level, timestamps, process identifiers, summary status indicators, and error detail messages. All error and status logs are formatted as flat files with a delimiter that makes it easy to import them into a database.

Senders and receivers are controlled by queue, port and input/output destinations which can be externally specified at instantiation. Multiple senders/receivers can thus be defined for multiple ports/destinations. The same sender-receiver pairs are used to transfer information from machine to machine, from queue to queue within a single machine, or from one collection of machines to another. In this way the multi-layered socket protocol of the invention supports internal communication as well as external communication between machines or clusters of machines whether on internal or external networks. Logs are collected for all of the processes. Each transfer socket is optimized for large binary records. Information sent through the socket protocol contains XML information about contained records and headers to improve efficiency.

The multi-layered socket protocol provides simultaneous transport of binary and text objects within XML streams, use of XML to specify message parameters and optionally contains summaries of binary content items, headers for version identification (indicative of the version of the protocol), message type indications (for application routing), message length indicators, and response packets for status information. The whole is carried on standard TCP/IP sockets optimized for large records, and large delay-bandwidth products. The multi-layered socket protocol also provides a flexible mechanism for transfer of queues of information from one location to another, including the ability to carry security tokens that authenticate endpoints.

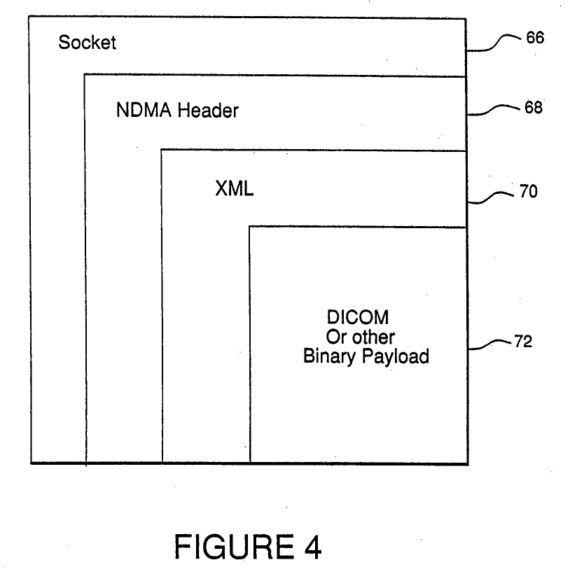

FIG. 4 is a diagram depicting the four layers 66, 68, 70, 72 of the multi-layered NDMA socket transport data structure (protocol) in accordance with an exemplary embodiment of the present invention. The multi-layered socket protocol implements a sender and receiver pair connected by sockets. All processes are multi-threaded (i.e. can process multiple records simultaneously). All processes create standard logs that are easily imported into databases. The multi-layered socket protocol provides for carrying security tokens that authenticate senders and receivers.

As illustrated, the multi-layered socket protocol comprises four layers; a socket layer 66, an NDMA header layer 68, an AL layer 70, and a binary record transport layer 72. The socket layer 66 supports versioning for backward compatibility, message IDs for tracking, and a response for send/receive status versification. The socket layer 66 also provides message type designators for rapid routing of message types and content. The NDMA Header layer 68 supports transport of both text and binary components within a message. A MIME-like structure with a text header and a binary payload is included in each message. The XML layer 70 carries sender and receiver information and authorization, length information and timestamps. The XML layer 70 contains unpacked critical data from the binary payload, constructed on the WallPlug 12. This information allows more rapid use of data avoiding time-consuming and/or repetitive unpacking of the large and complex binary payloads, such as found in DICOM payloads 72. The structure of the header can be used to detect machine endian-ness (i.e., whether the transmission's most significant bit is first or last). The XML message structures support a wide range of functions and are expandable. The XML message structures support reply structures that can be used to verify execution of other message functions.

As illustrated in FIG. 4, the structure of the transport protocol comprises nested layers. Standard TCPIP sockets are used at the top layer 66 with the ports selected from pre-approved sets of ports allowed by a firewall rule. The standard TCPIP socket carries the NDMA Header 68. This NDMA header 68 specifies the length of the message to follow, and the message type. The message type indicates whether the message contains DICOM related data or a binary payload. By introducing the message type indicator, the DICOM data may be sent in place of the binary payload of the XML. Upon receipt, the message type indicator is checked to determine if the payload contains DICOM or other binary image data or a conventional text payload. The NDMA header 68 can also contain length indicators for each subsection of the NDMA header 68 indicating the length of the content nested within the respective subsection, including the size of the nested DICOM or binary image data or the text data, depending upon the type of payload. Message types are used to identify content types without parsing the complete message and are used for rapid routing of messages within applications. The NDMA header 68 also specifies a version number for the protocol to provide backward compatibility. The NDMA header 68 also contains a message reference sequence number. The message reference sequence number can be used to associate the current message with a previous message, or messages. This can be used, for example, to indicate whether the current message is a response or acknowledgment to a previous message. The NDMA header 68 is expandable. For example, the NDMA header 68 can be expanded to add and/or update length indicators, message types, and/or version indicators.

The NDMA header 68 is followed by an XML layer 70. This XML layer 70 contains more detailed information about the particular message and may also contain information extracted from the DICOM or other binary packet 72 to follow. This is done to extract critical information needed by applications from the binary payload and to avoid having to unpack the full binary structure within each application. The XML layer 70 also carries sender and receiver information, point of origin identifiers, timestamps, and certificates. The XML layer 70 forms a virtual envelope which can be flexibly added to, providing useful information either to routing applications or to endpoint applications.

The XML layer 70 ends with a “PayLoad” indicator. The remainder of the message is assumed to be binary. This MIME-like structure with a text header and a binary payload allows the multi-layered protocol to pass both text and binary information without having to ASCII encode the binary data which is very inefficient and lengthens the message. This structure also allows the binary payload 72 (typically a binary DICOM image format or a binary DICOM Structured report but more generally any binary payload) to be passed bit-for-bit as it exists within the hospital/clinic without modification to the backend. The multi-layered socket protocol of the invention may also include a hash to be used later for tamper proof verification that the binary packet has never been modified. The protocol receivers/senders implement the automatic switching between ASCII and binary encoding methods. The payload section 72 can be of zero length for message structures without binary packets. For convenience, the NDMA header structure 68 is repeated in front of the binary information. Length indicators in the multi-layered socket protocol allow the receivers to be efficiently written and to be able to quickly test for completion of each portion of the transmission.

In an exemplary embodiment, the multi-layered socket protocol of the invention requires the receiver to transmit a 12 byte response which indicates the status (success/failure) of the transmission and storage at the receiving end. Socket port numbers used are typically 5000-5010 and 6000-6010, but the protocol can be used on any allowed port.

### Example Socket Protocol Header 66

2 bytes version number

2 bytes message type

2 bytes reserved

4 bytes content length

4 bytes message ID

### Example NDMA Header Structure 68

Header for an XML Segment

### NDMA/VERSION: 1.0

### CONTENT-TYPE: XML

### CONTENT-LENGTH: 761

XML text follows with length=761 bytes including <payload> tags

### NDMA/VERSION: 1.0

### CONTENT-TYPE: Image

### CONTENT-LENGTH: 8788864

(binary content follows with length=8788864 bytes)

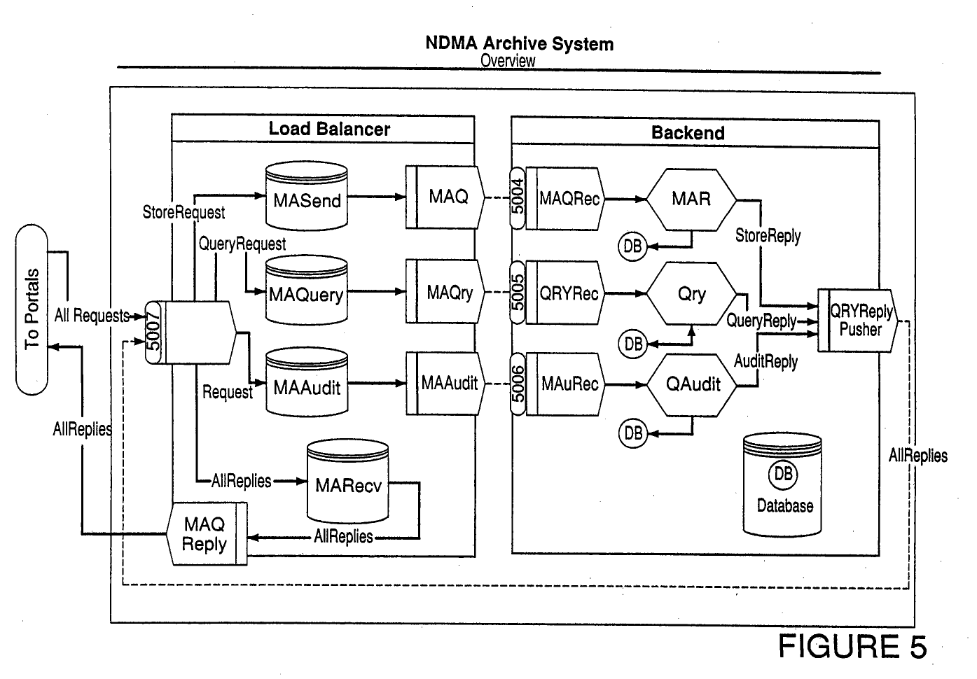

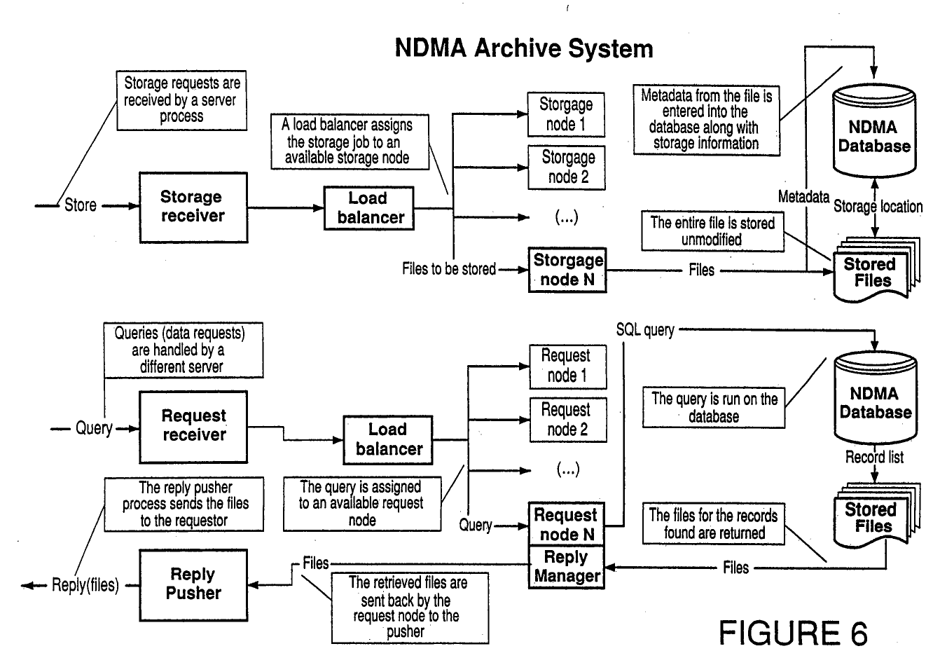

FIGS. 5 and 6 are diagrams of the backend systems of the NDMA Archive System, which depict an overview and the basic components of the NDMA Archive System, respectively. The multi-layered socket protocol is used for all information transfer indicated by arrows in FIGS. 5 and 6, typically between separate machines on an internal network. For a better understanding of FIGS. 5 and 6 herein, please refer to the related application entitled, “NDMA SCALABLE ARCHIVE HARDWARE/SOFTWARE ARCHITECTURE FOR LOAD BALANCING, INDEPENDENT PROCESSING, AND QUERYING OF RECORDS”, Attorney Docket UPN-4382/P3189, filed on even date herewith, the disclosure of which is hereby incorporated by reference in its entirety.

Three features of the multi-layered socket protocol allow the backend systems to rapidly and easily route information of different types to processes or to queues for handling specific data types. First, a dedicated socket number can be used for any protocol, and senders and receivers can be connected to sockets with a unique port number. Second, information in the message type designator in the protocol can be used to separate traffic of different types arriving on a single port to trigger special processing for certain types of records. Finally, XML content extracted from the original DICOM or other binary object 72 and placed in the XML section 70 of the protocol can be used whenever information from the binary object is required, but when it is too time consuming or inconvenient to unpack the object itself.

In an exemplary embodiment, the type designators have the following functions.

Type 0 query for clinical records

Type 1 reply to query

Type 2 store image request

Type 3 HIPPA Audit store request

Type 4 query for research records

Type 5 Forward query result to image owner node

Type 9 Fetch Research Image and de-identify

Example sockets include:

5004 send store request to backend

5005 send query to backend, route forward request to backend

5006 send audit record to backend

5007 receiver from portals, sender to backup, receiver for replies

5008 receive reply from query

6007-8 test and heartbeat records

Example XML Structure

<?xml version=“1.0” encoding =“UTF-8” ?>

<Message type=“StoreImage”>

<MessageID>

<OriginalIP>130.91.51.20</OriginalIP>
<Timestamp>5/12/2003 9:00:01 AM</Timestamp>
<MessageNum>-13432</MessageNum>
</MessageID>

<Request action=“Store” type=“Image”>

<ID>-13432</ID>
<Priority>Routine</Priority>
</Request>

<Sender>

<Certificate>BB9118189xxxxxxxxx92D985DEB7C29</Certificate>
<Requestor>

<Facility>NSCP</Facility>
<ID>NSCP-6007</ID>

</Requestor>

</Sender>
<Receiver>

<Certificate>BB9118189xxxxxxxxx92D985DEB7C29</Certificate>
<IP>130.91.51.20</IP>

</Receiver>
<Payload>

<Record type=“Image” format=“DICOM”>

<Item>

<Name>PatientID</Name>
<Value>pid_745566</Value>

</Item>

<Item>

<Name>NamePatientFull</Name>
<Value>dummy_745566</Value>
</Item>

</Record>

</Payload>

</Message>

Example Message with NDMA Headers, and Both XML and Binary Data

NDMA/VERSION: 1.0
CONTENT-TYPE: XML
CONTENT-LENGTH: 761
<?xml version=“1.0” encoding=“UTF-8”?>
<Message type=“StoreImage”>
<MessageID>
<OriginalIP>192.168.201.1</OriginalIP>
<Timestamp>1052162767</Timestamp>
<MessageNum>9953</MessageNum>
</MessageID>
<Sender>
<Certificate>F966175489xxxxxxxx38F37112E3</Certificate>
<Requestor>
<Facility>ORDEV</Facility>
<ID>IP134167143162</ID>
</Requestor>
</Sender>
<Receiver>
<Certificate>0CF4AD709xxxxxxxxxxxB0BF8C4</Certificate>
<Ip>130.91.50.15</Ip>
</Receiver>
<Request action=“Store” type=“Image”>
<ID>9953</ID>
<Priority>LOW</Priority>
</Request>
<Payload>
<Record type=“Image” format=“DICOM”>
<Item>
<Name>PatientID</Name>
<Value>99990032</Value>
</Item>
<Item>
<Name>NamePatientFull</Name>
<Value>xxxxx\hat{ }xxxxx</Value>
</Item>
</Record>
</Payload>
</Message>
NDMA/VERSION: 1.0
CONTENT-TYPE: Image
CONTENT-LENGTH: 8788864
(binary content follows with length = 8788864 bytes)

Application Layer Headers

### Example Socket Header 66 Format

| Element | Length | Description |
|---|---|---|
| Version | 2 bytes | NDMA Version |
| Message Type | 2 bytes | Type of Message |
| Length | 4 bytes | Length of message in bytes |
| Request (Message) ID | 4 bytes | Unique identifier created at portal |

### Socket Message Types

| | |
|---|---|
| QueryClinical | 0 |
| Reply | 1 |
| StoreImage | 2 |
| StoreAudit | 3 |
| QueryResearch | 4 |
| RequestCAD | 6 |
| RequestAuthentication | 7 |
| StoreAuthList | 8 |
| Acknowledgement | 100 |
| Negative Acknowledgement | 101 |
| StoreDSRNMD | 501 |
| StoreDSRMMM | 502 |
| StoreDSRANNOT | 503 |
| FetchResearch | 901 |
| FetchClinical | 902 |

### Example NDMA Header 68 Format

### NDMA/VERSION: 1.0

### CONTENT-TYPE: XML

CONTENT-LENGTH: nnnnn

### Example NDMA XML 70, 72 Message Structures

The following table lists example messages and details about the messages.

| | | | Socket |
|---|---|---|---|
| Message | Message Description | Detail | Message Type |
| Hospital Linux | | | |
| to Archive | | | |
| L-A 1 | Request for clinical records | XML only | 0 |
| L-A 2 | Request for Research Records | XML only | 4 |
| L-A 3 | Request for Audit Records | XML only | |
| L-A 4 | Request to Store DICOM Image | XML + binary | 2 |
| L-A 5 | Request to Store DICOM NMD SR | XML + binary | 501 |
| L-A 6 | Request to Store DICOM MMM SR | XML + binary | 502 |
| L-A 7 | Request to Store DICOM Annotation SR | XML + binary | 503 |
| L-A 8 | Request to Store HIPAA Audit | XML only | 3 |
| L-A 9 | Request to Store MAP Audit | XML + binary | 3 |
| L-A 10 | Request to Fetch Research Records | XML only | 901 |
| L-A 11 | Request to Fetch Clinical Records | XML only | 902 |
| Archive to | | | |
| Hospital Linux | | | |
| A-L 1a | Reply with records to request for clinical | XML + binary | 1 |
| | records | | |
| A-L 1b | Reply with records to request to fetch | XML + binary | 1 |
| | research records | | |
| A-L 2 | Reply with Status (to request to store) | XML only | 1 |
| A-L 3 | Reply with Status (to request for | XML only | |
| | records that returned no data | | |
| A-L 4 | Reply with Records (to request for Audit | XML only | |
| | Data) | | |
| A-L 5 | Reply to initial Research Request/Query | XML + binary | 1 ? |
| Windows to | | | |
| Hospital Linux | | | |
| W-L 1 | Request to Store DICOM Image | XML + binary | 2 |
| W-L 2 | Request to Store DICOM NDM SR | XML + binary | 501 |
| W-L 3 | Request to Store DICOM MMM SR | XML + binary | 502 |
| W-L 4 | Request to Store DICOM Annotation SR | XML + binary | 503 |
| W-L 5 | Request to Store MAP Audit | XML + binary | 3 |
| W-L 6 | Reply with Status | XML only | 1 |
| | | (response to | |
| | | request to move | |
| | | records to | |
| | | hospital) | |
| W-L 7 | Request Authentication | XML only | 7 |
| W-L 8 | Request CAD | XML only? | 6 |
| Hospital Linux | | | |
| to Windows | | | |
| L-W 1 | Request to Move Records into Hospital | XML + binary | |
| L-W 2 | Reply with Status | XML only | 1 |
| | | (response to | |
| | | request to store) | |
| L-W 3 | | XML only | 1 |
| | | (response to | |
| | | request for | |
| | | authentication) | |
| L-W 4 | Request to Store DICOM Device | XML only | 8 |
| | Authentication List | | |

A multi-layered NDMA socket transport protocol in accordance with the present invention enables intra-enterprise and/or inter-enterprise data exchange for hospital records. The multi-layered NDMA socket transport protocol enables the interaction of dissimilar, multi-vendor, geographically distributed and heterogeneous hardware systems within hospitals for records transfer, storage, searching, and processing. In particular, the multi-layered NDMA socket transport protocol links WallPlug-type devices to NDMA Archive System resources.

The multi-layered NDMA socket transport protocol also links internal Archive communications. Thus, archive operations can be distributed geographically and implemented on heterogeneous systems or can be implemented on single computers, or on collections of computers.

Although illustrated and described herein with reference to certain specific embodiments, the present invention is nevertheless not intended to be limited to the details shown. Rather, various modifications may be made in the details within the scope and range of equivalents of the claims and without departing from the invention.

What is claimed:

1. A multilayered data structure for communicating binary image data between a device that generates the binary image data and a remote NDMA archive system for storage of the binary image data, said structure comprising:
a first layer comprising a socket protocol;
a second layer nested within said first layer, said second layer comprising a national digital mammography archive (NDMA) header;
a third layer nested within said second layer, said third layer comprising extensible markup language (XML) text; and
a fourth layer nested within said third layer, said fourth layer comprising said binary image data.

2. A data structure in accordance with claim 1, wherein said NDMA header comprises at least one of a version indicator indicative of a version of said data structure, a type indicator indicative of a type of said binary image data, a content length indicative of a content length of said third and fourth layers, and a message reference sequence number.

3. A data structure in accordance with claim 2, wherein said type indicator is indicative of whether said binary image data comprises Digital Imaging and Communications in Medicine (DICOM) related data, a binary payload, or text.

4. A data structure in accordance with claim 2, wherein said binary image data is routed in accordance with said type indicator.

5. A data structure in accordance with claim 2, wherein said message reference sequence number is indicative of whether a content of said data structure is a reply message.

6. A data structure in accordance with claim 2, wherein said NDMA header is expandable to at least one of add and update, at least one of said version indicator, said type indicator, said content length, and said message reference sequence number.

7. A data structure in accordance with claim 2, wherein said data structure provides, via said version indicator, backward compatibility for future data protocol modifications.

8. A data structure in accordance with claim 1, wherein said NDMA header comprises a length indicator for each subsection of said NDMA header indicative of a length of the content nested within a respective subsection.

9. A data structure in accordance with claim 1, wherein said XML text comprises at least one of routing information and application specific information.

10. A data structure in accordance with claim 1, wherein said fourth layer comprises a non-ASCII encoded binary image payload.

11. A data structure in accordance with claim 1, wherein said first layer is compatible with a Transmission Control Protocol/Internet Protocol (TCP/IP).

12. A data structure in accordance with claim 1, wherein said first layer is compatible with a Transmission Control Protocol/Internet Protocol (TCP/IP) comprising at least one header and a protocol response.

13. A data structure in accordance with claim 1, wherein said NDMA header comprises a type indicator indicative of at least one of how said data structure is to be processed and how said data structure is to be routed.

14. A data structure in accordance with claim 1, wherein said NDMA header comprises selected portions of said XML text.

15. A data structure in accordance with claim 14, wherein said selected portions comprise at least one of routing information, timing information, identification information, extracted DICOM related information, and binary data.

16. A data structure in accordance with claim 1, wherein said binary image data comprises a message having non-encoded text and binary image data therein.

17. A data structure in accordance with claim 1, wherein said binary image data comprises at least one of Digital Imaging and Communications in Medicine (DICOM) image data and DICOM structured report data.

18. A method for transferring binary image data between a digital imaging and communications in medicine (DICOM) compatible device and a storage device, wherein said binary image data comprises one of DICOM related data and a binary payload, said method comprising the steps of:
opening a socket and sending a socket protocol header indicating a total number of bytes to follow;
sending a first NDMA header for content type XML, each NDMA header containing version and length specifiers;
sending an XML message containing message identifiers, requested actions, and sender and receiver specifications;
sending a second NDMA header for content type binary image data; and
sending a binary payload containing the binary image data.

19. The method of claim 18, comprising the step of sending NDMA headers and associated content until the total number of bytes specified in said socket protocol header have been sent.

20. The method of claim 18, wherein said sender and receiver specifications include authentication data for authenticating at least one of a sender and a receiver of said binary image data.

21. The method of claim 18, wherein said XML message includes data associated with binary image data in a binary payload to follow said XML message, including the step of software applications processing said data associated with said binary image data.

22. The method of claim 18, said version specifier enables backward compatibility for future data protocol modifications.

23. The method of claim 18, further comprising the step of sending an XML message containing a reply message identifier, requested actions, and sender and receiver specifications but no binary payload for a reply acknowledgement.
